(19) United States
(12) Patent Application Publication
(10) Pub. No.: US 2015/0141673 A1
(43) Pub. Date: May 21, 2015

(54) NOVEL CRYSTALLINE FOR W OF CABAZITAXEL AND METHOD FOR PREPARING IT

(71) Applicants: Honghai Song, Tianjin (CN); Xingfeng Wang, Tianjin (CN); Song Lin, Tianjin (CN)

(72) Inventors: Honghai Song, Tianjin (CN); Xingfeng Wang, Tianjin (CN); Song Lin, Tianjin (CN)

(21) Appl. No.: 14/080,990

(22) Filed: Nov. 15, 2013

(51) Int. Cl. C07D 305/14

(52) U.S. Cl. CPC C07D 305/14
USPC 549/510

(57) ABSTRACT

The present invention relates to a novel crystalline polymorph form W of cabazitaxel and to method for the preparation thereof.

The present application claims priority to Chinese Patent Application No. 201310440458.4, filed on Sep. 24, 2013.

### BACKGROUND OF THE INVENTION

Cabazitaxel belongs to the taxane class and is closely related in both chemical structure and mode of action to the anticancer drugs paclitaxel and docetaxel. Cabazitaxel, chemically known as (2α, 5β7β, 10β, 13α)-4-acetoxy-13({2,3S)-3-[(tert-butoxycarbonyl)amino]-2-hydroxy -3 -phenylpropanoyl}oxy)-1-hydroxy-7,10-dimethoxy-9-oxo-5,20-epoxy-tax -11-en-2-yl benzoate, is represented by formula (I).

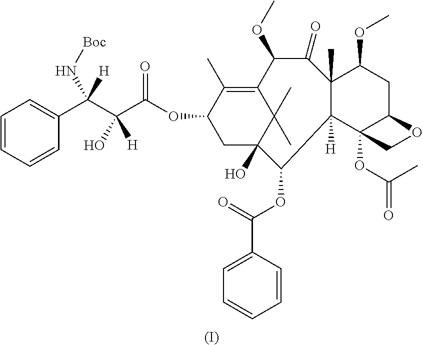

Cabazitaxel is microtuble inhibitor, which can promote the formation of tubulin by combined with tubulin protein, while cabazitaxel also an prevent the mierotubule assembled to disassemble.

Crystalline form is one of the important factors to affect the drug quality, drug efficacy and performance of pharmaceutic preparation. In view of the good effect of cabazitaxel, the people have made a lot of research on it and developed many crystalline form. Finally, crystalline acetone solvate form of (2α, 5β, 7β, 10β, 13α)-4-acetoxy -13-({2,3 S)-3-[(tert-butoxycarbonyl)amino]-2-hydroxy-3phenylpropanoyl}oxy)-1-hydroxy-7,10-dimethoxy-9-oxo-5,20-epoxy-tax-11-en-2-yl benzoate is known and described in WO2005028462, which is named crystalline form A. Followed crystalline forms B, C, D, E, F are known and described in Chinese patent CN101918385, which are all converted from form A. While crystalline form B, D, E, F ethanol solvates are also known and described in patent CN101918385.

In summary, as we know, the preparation of crystalline forms of cabazitaxel needs specific environment to realize, which is had to industrialize. Further more, in the process of the preparation of crystalline form, we can find high temperature has been used to remove the solvent which makes the purity of the product to decrease. It is also known that the ability of a substance to exist in more than one crystal form is defined as polymorphism and its different crystal forms are called polymorphs. Polymorphs will affect the solubility, stability and bioavailability of drug, furthermore it will affect the quality, safety, effectiveness and application of drug.

### BRIEF SUMMARY OF THE INVENTION

In one aspect, the present invention provides a crystalline form of cabazitaxel. The novel form have chemically characterized by ¹HNMR (nuclear magnetic resonance) spectroscopy, XRPD, FTIR (Fourier transform infrared) spectroscopy (also abbreviated to IR spectroscopy), TGA (theramgravimetric analysis) DSC (differential scanning calorimetry).

In second aspect, the present invention provides the method of preparations for the novel crystal line form.

In third aspect, the present invention provides process for the preparation of the crystalline form W of cabazitaxel. In some embodiments, the inventive process includes:

a) any kind of crystalline form of cabazitaxel dissolves in appropriate organic solvent, appropriate ionized water is added slowly, after finished dropwise the solution is kept at 20-25° C. for 16 hours, then colorless crystal formed;

b) filtering the solid and washed the solid with methanol (50%);

c) drying the isolated and washed solid resulting from step b) under vacuum, at 40-50° C.;

### BRIEF DESCRIPTION OF THE DRAWINGS

FIG. 1 shows the XRPD pattern for cabazitaxel Form W.

FIG. 2 shows the DCS trace of cabazitaxel Form W.

### DETAILED DESCRIPTION OF THE INVENTION

The present invention provides novel crystalline form W of cabazitaxel. The crystalline forms can be produced by method described herein in substantially pure form, i.e., at least with 80% purity or higher. Form W can be produced with a purity of at least 90%, preferably at least 95% and more preferably at least 98% and the most preferably at least 99%.

In one aspect, the present invention provides a crystalline form of (2α, 5β, 7β, 10β, 13α)-4-acetoxy-13-({(2,3 S)-3-[(tert,butoxycarbonyl)amino]-2-hydroxy-3-phenylpropanoyl}oxy)-1-hydroxy-7, 10-dimethoxy-9-oxo-5,20-epoxy-tax-11-en-2-yl benzoate.

The crystalline compound of the present invention can be characterized by a number of techniques including X-ray power diffraction (XRPD), infrared spectroscopy (IR), differential scanning calorimetry (DSC), thermal gravimetric analysis (TGA), and crystallography.

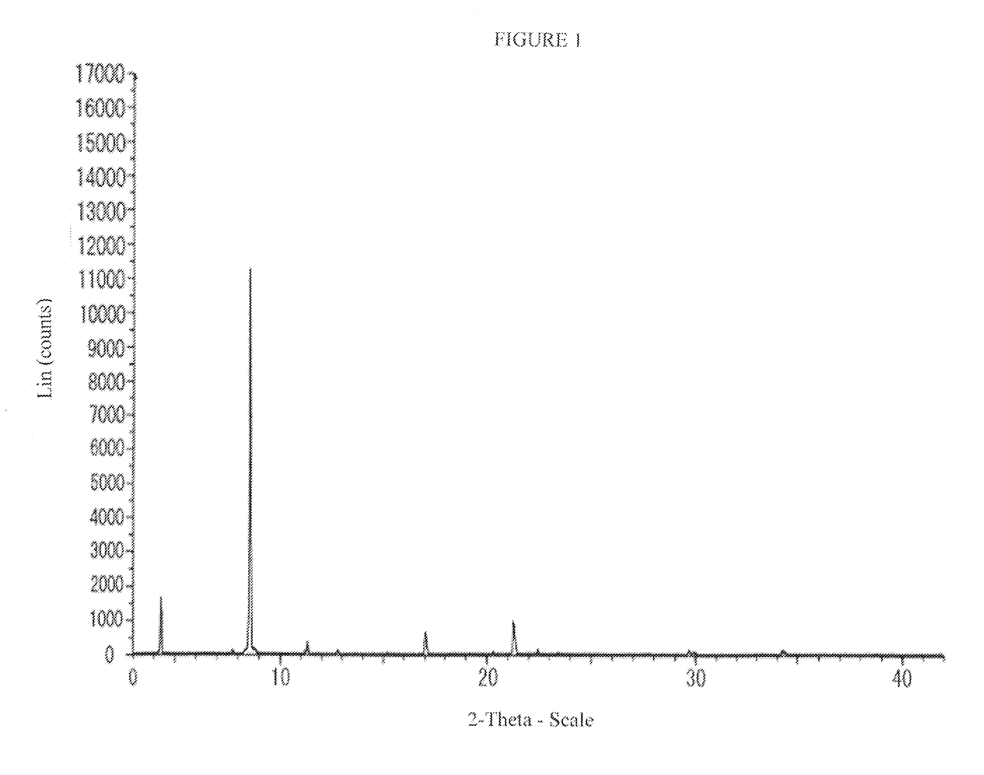

In some embodiments, the present invention provides the crystalline form of the compound characterized by an XRPD pattern substantially in accordance with that of FIG. 1.

In other embodiments, the crystalline form of the compound is Form W. characterized by XRPD pattern that includes one or more peaks at 4.4, 7.8, 8.5, 11.4, 12.8 15.3, 17.0, 20.4, 21.4, 22.5, 23.4, 27.8, 29.7, 29.9, 33.3 and 34.3 degrees 2θ (±0.2 degrees 2θ). Wherein said XRPD patter is made using CuKα₁ radiation. Preferably, the XRPD pattern shall have at least the following peaks 4.4, 8.5, 17.0 and 21.4 degrees.

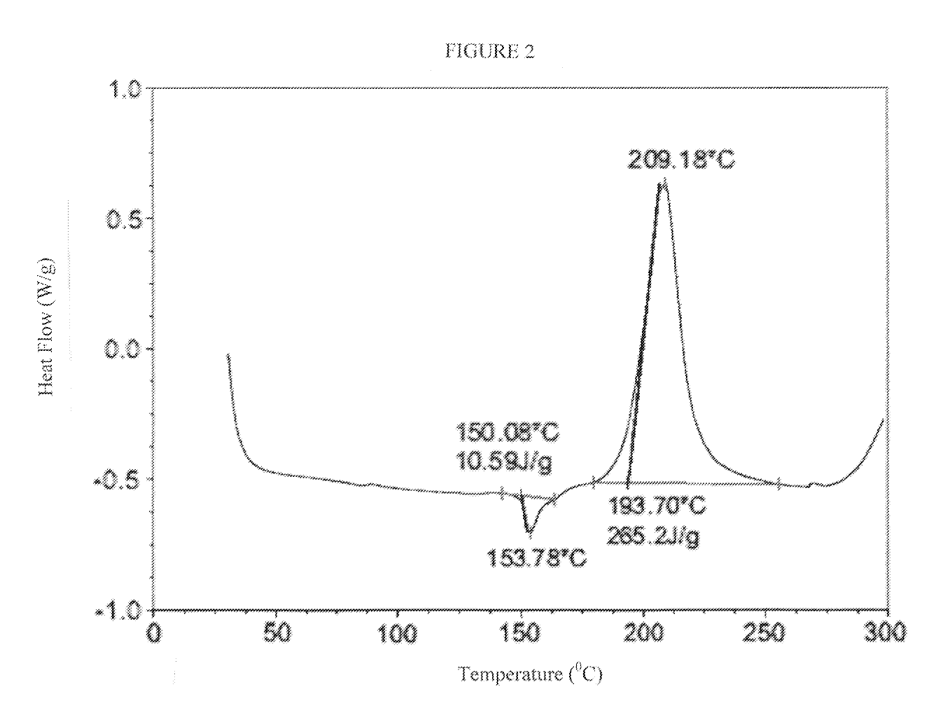

Crystalline Form W of the present invention is also characterized by a DSC substantially in accordance with FIG. 2.

In a related aspect, the present invention provides a process for preparing crystalline Form W of cabazitaxel including:

a.) any kind of crystalline form of cabazitaxel dissolves in appropriate organic solvent, appropriate ionized water is added slowly, after finished drop wise the solution is kept at 20-25° C. for 16 hours, then colorless crystal formed;

b) filtering the solid and washed the solid with methanol (50% aqueous);

c) drying the isolated and washed solid resulting from step b) under at 40-50° C.

The organic solvent above-mentioned include methanol, ethanol, isopropanol and acetonitrile etc. Appropriate ionized water is added slowly, while keep the volume radio of organic solvent and water at the range of 55:45-85:15. The temperature of the solution must be kept at 15-25° C. during the crystallizing. The drying time should be kept below 24 hours and the temperature should be kept at 40-50° C.

The present invention will be described more fully by means a the following examples which should not be considered to limit the invention.

Experimental analysis conditions:
Differential scanning calorimetry (DSC):

The measurements were carried out on a T.A. The sample is subjected to temperature programming from 30° C. to 300° C. with a heating rate of 5° C./min. The product was placed in a crimped aluminum capsule and the amount of product analyzed is between 2 and 5 mg.

### Power X-ray Diffraction (PXRD)

The analyses were carried out on a Panalytical X'Pert Pro diffractometer with a reflection-mode Braff-Brentano focusing geometry (θ-2θ) assembly. The product analyzed is deposited as a thin layer on a silicon single crystal. A copper anticathode tube (45 kV/40 mA) supplies an incident radiation Cu-Kα₁) (λ=1.54056). The beam is collimated using Sollers slits which improve the parallelism and variable slits which limit scattering. An X'celerator detector completes the device. The diagram recording characteristics are the following: sweeping from 2 to 40 degree, 0.01°/1sec.

### EXAMPLES

The following examples are provided to further illustrate, but not to limit this invention.

### Example 1

Cabazitaxel (500 mg) was dissolved in 10 mL of methanol, and then ionized water (5 mL) was dropwised into it with stirring. Then the solution was kept at 15-25° C. for 16 hours. Then the mixture was filtered, and the collected solids were washed with methanol (50% aqueous) and dried in vacuum at 40° C. for 24 hours to give cabazitaxel Form W (490 mg, purity: 99.6%) as colorless solid (melting point 153.78 ° C).

### Example 2

Cabazitaxel (500 mg) was dissolved in 10 mL of ethanol, and then ionized water (5 mL) was dropwised into it with stirring. Then the solution was kept at 15-25° C. for 16 hours. Then the mixture was filtered, and the collected solids were washed with ethanol (50% aqueous) and dried in vacuum at 40° C. for 24 hours to give cabazitaxel Form W (492 mg, purity: 99.2%) as colorless solid.

### Example 3

Cabazitaxel (500 mg) was dissolved in 10 mL of IPA, and then ionized water (5 mL) was dropwised into it with stirring. Then the solution was kept at 15-25° C. for 16 hours. Then the mixture was filtered, and the collected solids were washed with IPA (50% aqueous) and dried in vacuum at 40° C. for 24 hours to give cabazitaxel Form W (460 mg, purity: 99.3%) as colorless solid.

### Example 4

Cabazitaxel (500 mg) was dissolved in 10 mL of acetonitrile, and then ionized water (5 mL) was dropwised into it with stirring. Then the solution was kept at 15-25° C. for 16 hours. Then the mixture was filtered, and the collected solids were washed with acetonitrile (50% aqueous) and dried in vacuum at 40-50° C. for 24 hours to give cabazitaxel Form W (482 mg, purity: 98.9%) as colorless solid.

powder X-ray diffraction and DSC data on form W is summarized in the following Table 1 and compared with existing polymorphs:

TABLE 1

| Form | Patent | Characteristic peak | mp (° C.) |
|---|---|---|---|
| Anhydrous B | CN101918385A | 7.3, 8.1, 9.8, 10.4, 11.1, 12.7, 13.1, 14.3, 15.4 | 150 |
| Anhydrous C | CN101918385A | 4.3, 6.8, 7.4, 8.7, 10.1, 11.1, 11.9, 12.3, 12.6, | 146 |
| | | 13.1 | |
| Anhydrous D | CN101918385A | 3.9, 7.7, 7.8, 7.9, 8.6, 9.7, 10.6, 10.8, 11.1, | 175 |
| | | 12.3 | |
| Anhydrous E | CN101918385A | 7.1, 8.1, 8.9, 10.2, 10.8, 12.5, 12.7, 13.2, 13.4, | 157 |
| | | 13.9 | |
| Anhydrous F | CN101918385A | 4.4, 7.2, 8.2, 8.3, 8.8, 9.6, 10.2, 10.9, 11.2, 12.1 | 148 |
| | | 12.3 | |
| Ethanol | CN101918385A | 7.3, 7.8, 8.8, 10.2, 12.6, 12.9, 13.4, 14.2, 14.7, | N/A |
| solvate B | | 15.1 | |
| Ethanol | CN101918385A | 3.8, 7.5, 7.7, 8.4, 9.4, 10.3, 10.5, 11.1, 11.5, | N/A |
| solvate D | | 11.9 | |
| Ethanol | CN101918385A | 7.1, 8.1, 8.8, 10.2, 10.7, 12.5, 13.2, 13.4, 13.9, | N/A |
| solvate E | | 14.2 | |
| Ethanol water | CN101918385A | 4.4, 7.2, 8.2, 8.3, 8.8, 9.6, 10.3, 10.9, 11.2, | N/A |
| F | | 12.2 | |
| Monohydrate | CN101918385A | 4.3, 6.8, 7.4, 8.6, 10.1, 11.1, 11.9, 12.2, 12.6, | N/A |
| C | | 13.3 | |
| Dihydrate C | CN101918385A | 4.2, 6.9, 7.5, 8.4, 9.9, 10.9, 11.7, 12.3, 12.6, | N/A |
| | | 13.2 | |
| Anhydrous | CN102675257A | 4.3, 7.1, 8.7, 10.2, 10.9, 12.2, 13.8, 15.2, 16.4, | N/A |
| solvate | | 17.0, 17.6, 18.3, 19.2, 19.6, 20.3 | |
| Ester solvate | CN102746258A | 7.9, 8.5, 10.1, 12.6, 14.0, 15.0, 15.8, 17.3, | N/A |
| J | | 19.4, 20.1 | |
| Hydrate G | CN102746258A | 4.5, 8.5, 8.9, 11.1, 12.4, 13.9, 15.4, 17.7, 19.3 | N/A |
| Form 1 | CN102746258A | 7.4, 7.8, 8.9, 10.1, 14.4, 15.0, 15.7, 17.7, 19.6, | N/A |
| | | 23.5 | |
| IPA solvate | WO2013069027 | 7.4, 7.9, 8.9, 10.3, 12.6, 13.3, 14.4, 15.2, 16.5, | 156.98 |
| | A1 | 17.0, 17.7, 18.3, 19.5, 20.5 | |
| Form 1 | WO2013080217 | 7.3, 8.1, 8.9, 9.8, 10.4, 11.1, 12.7, 14.3, 15.3, | 134.01, |
| | A2 | 15.8 | 159.58 |
| Form 2 | WO2013080217 | 3.9, 6.9, 7.8, 10.2, 10.7, 11.6, 12.2, 12.8, 13.6, | 68.5, |
| | A2 | 14.0, 15.1, 17.2, 18.1 | 114.9, |
| | | | 174 |
| Form 3 | WO2013080217 | 4.2, 6.9, 7.5, 8.5, 8.6, 10.1. 11.0, 11.8, 12.3, | 71.59 |
| | A2 | 12.6, 13.3, 13.4, 13.8, 14.3, 35.1, 15.6 | |
| Form 4 | WO2013080217 | 6.9, 7.9, 10.2, 10.7, 12.2, 13.9, 15.1, 17.2, | 45.35, |
| | A2 | 18.1, 19.8 | 124.92 |
| Form 5 | WO2013080217 | 7.5, 7.9, 8.6, 10.0, 12.6, 13.3, 14.1. 14.8, 15.0, | 56.29, |
| | A2 | 15.8, 16.7, 17.4, 18.0, 18.8, 19.4, 20.1 | 145.27 |
| Form 6 | WO2013080217 | 7.5, 7.9, 8.6, 9.0, 10.0, 12.5, 13.2, 13.8, 14.1, | 157.26 |
| | A2 | 15.0, 15.5, 15.8, 16.6, 17.3, 17.8, 18.8, 19.6, | |
| | | 20.1 | |
| Form 7 | WO2013080217 | 5.2, 6.0, 7.5, 8.9, 9.5, 10.1, 10.7, 11.7, 12.1, | 162 |
| | A2 | 12.8, 13.3, 14.1, 15.3, 16.1, 17.1, 17.6, 18.1, | |
| | | 18.9, 19.6 | |
| Form 8 | W02013080217 | 7.5, 7.9, 8.6, 10.0, 12.6, 13.3, 14.1, 14.8, 15.8, | 151.84, |
| | A2 | 16.7, 17.0, 17.5, 18.0, 18.9, 19.4 | 159.08 |
| Form 9 | WO2013080217 | 7.5, 7.9, 15.0, 15.8, 18.1, 19.4, 20.1, 22.6 | 156.51 |
| | A2 | | |
| Form 10 | WO2013080217 | 7.4, 7.7, 8.8, 10.1, 12.2, 12.7, 13.2, 14.4, 15.3, | 163.24 |
| | A2 | 16.2, 16.9, 17.6, 18.0, 18.7, 19.4 | |
| Form 11 | WO2013080217 | 7.4, 7.7, 8.8, 10.1, 12.2, 12.7, 13.2, 14.4, 14.8, | 162.26 |
| | A2 | 15.3, 15.6, 16.2, 16.9, 17.6, 18.0, 18.4, 19.4 | |
| Form 12 | WO2013080217 | 7.2, 7.7, 8.1, 8.9, 9.8, 10.3, 11.0, 12.6, 13.2, | 150.47 |
| | A2 | 14.2, 14.8, 15.1, 15.7, 16.3, 17.1, 17.5, 18.5, | |
| | | 19.4, 19.8 | |
| Form 13 | WO2013080217 | 7.6, 8.2, 8.9, 9.7, 10.2, 10.9, 11.7, 12.6, 13.1, | 119.57 |
| | | 13.6, 14.5, 15.1, 15.8, 16.2, 17.0, 17.8, 18.3, | |
| | | 19.2, 19.9 | |
| Ethyl acetate | WO2013088335 | 7.5, 7.9, 8.7, 10.1, 10.2, 12.6, 12.9, 13.8, | N/A |
| solvate | A1 | 14.1, 14.8 | |
| Form W | Present invention | 4.4, 7.8, 8.5, 11.4, 12.8, 15.3, 17.0, 20.4, 21.4 | 153.78 |
| | | 22.5, 23.4, 27.8, 29.7, 29.9, 33.3, 34.3 | |

1. (canceled)

2. (canceled)

3. (canceled)

4. A crystalline polymorph form W of cabazitaxel, having X-ray powder diffraction pattern substantially in accordance to FIG. 1, wherein said form W of cabazitaxel has a melting point of about 150° C.

5. The crystalline polymorph form W of claim 4, wherein said form W is substantially pure.

6. The crystalline polymorph form W of claim 4, wherein said form W is at least 90% pure.

7. The crystalline polymorph form W of claim 4_1 wherein said form W is at least 95% pure,

8. The crystalline polymorph form W of claim 4, wherein said form W is at least 98% pure,

9. The crystalline polymorph form W of claim 4, wherein said form W is at least 99% pure.

10. A process for preparing crystalline polymorph form W of comprising the steps of:
(a) dissolving cabazitaxel in methanol;
(b) adding water to the dissolved cabazitaxel in step (a) dropwise;
(c) keeping the solution of step b) at 20-25° C. for 16 hours to form colorless crystal:
(d) filtering the mixture of step (c) to get a colorless crystal;
(e) washing the colorless crystal with 50:50 methanol-water mixture;
(f) drying the colorless crystal from step (e) 40-50° C. to get said crystalline polymorph form W, wherein said form W of cabazitaxel has a inciting point of about 150° C.

11. (canceled)

12. The process of claim 10, wherein said water is added while keeping the volume radio of methanol and water at the range of 55:45-85:15.

13. The process of claim 10, wherein said the temperature of the solution is at 15-25° C. during the crystal wherein the drying time should be kept below 24 hours and the temperature is kept at 40-50° C.

14. The crystalline polymorph form W of cabazitaxel, wherein said form W of cabazitaxel is further characterized by DSC substantially in accordance with FIG. 2.
